(19) United States
(12) Patent Application Publication
(10) Pub. No.: US 2025/0276346 A1
(43) Pub. Date: Sep. 4, 2025

(54) SYSTEMS AND METHODS FOR SANITIZATION OF STETHOSCOPE HEADS

(71) Applicant: GG INNOVATIONS LLC, Chester, NJ (US)

(72) Inventor: GianAngelo GRACI, Cedar Knolls, NJ (US)

(21) Appl. No.: 19/069,546

(22) Filed: Mar. 4, 2025

Related U.S. Application Data
(60) Provisional application No. 63/560,843, filed on Mar. 4, 2024.

(51) Int. Cl. B08B 1/14; A61B 7/02; A61L 2/18; A61L 2/24; B08B 1/30; B08B 1/40

(52) U.S. Cl. CPC B08B 1/143; A61B 7/02; A61L 2/18; A61L 2/24; B08B 1/30; B08B 1/40; A61L 2202/123; A61L 2202/14; A61L 2202/17; A61L 2202/24

(57) ABSTRACT

A device for sanitizing stethoscope heads is disclosed. The apparatus includes a base adapted to be secured to an existing sanitation cloth dispenser. The device further includes a wipe plate extending from the base, an actuator, and motor that draws the sanitation cloth from the dispenser in response to actuation of the actuator.

### BACKGROUND

### Field of the Disclosure

The present disclosure generally relates to systems and methods for sanitizing stethoscope heads. More particularly, the present disclosure relates to systems and methods for dispensing sanitization cloths from a pre-existing container of sanitization cloths and arranging the sanitization cloths for convenient sanitization of the stethoscope head.

### Description of Related Art

Hospitals and other medical facilities require regular sterilization or disinfection of medical instruments, such as stethoscopes. Containers of sanitization cloths are commonly used in medical facilities to sterilize various medical devices and other objects in these facilities. Typically, the sanitization cloths are provided as a continuous roll within the container and the continuous roll of sanitization cloths is provided with perforations therealong to allow for tearing or separating one or more of the sanitization cloths from the continuous roll as desired. In many medical facilities, the sanitization cloths containers are disposed within a mounting bracket secured to a wall. Furthermore, a typical sanitization cloths container includes a top portion with an aperture to allow access to the interior of the container for drawing the sanitization cloths therefrom and a resealable lid to prevent sanitization fluid of the sanitization cloths from evaporating through the aperture. FIG. 1 shows a typical sanitization cloths container 1 with a lid 2 and a continuous roll of sanitization cloths disposed therein. FIG. 2 shows a typical bracket 5 for mounting the container 1 on a wall or other surface of a medical facility.

However, current systems for dispensing and utilizing the sanitization cloths present several disadvantages. For example, these systems require one or more of the sanitization cloths to be removed or torn from the continuous roll before using the cloths to sanitize or disinfect an object such as a stethoscope. As such, a user must touch (e.g., grasp) the cloths before using them for sanitization, thereby reducing the effectiveness of sanitization. Furthermore, some sterilization cloths contain sterilization fluid comprising chemicals hazardous to human skin, such that medical personnel and other users are required to put on (e.g., disposable) gloves or the like prior to grasping the cloths, thereby adding even more difficulty and complexity to current processes for stethoscope sanitization. Additionally, the removal or tearing of the cloths is a manual process requiring several steps, such as, opening the lid of the container, dispensing one or more sanitization cloths from the container, tearing the one or more cloths from the continuous roll along the perforations, and resealing the lid of the container. This manual process wastes valuable medical professional's time and can lead to sanitization cloths being wasted, for example, due to excess sanitization cloths being drawn from the container or not separated along the intended perforation line, or due to the lid of the container being left open or ajar, thereby allowing the sanitization fluid to evaporate and the sanitization cloths to dry out. Further still, after the sanitization cloth is used, it must be disposed of in a suitable waste receptacle. Because the foregoing process for dispensing sanitization cloths requires a plurality of cumbersome manual steps, which may require a medical professional to divert their attention from a patient in order to complete, the sanitization cloths are often times not utilized, causing stethoscopes and other medical devices to be sterilized at a less than desired, and less than the CDC recommended, frequency.

Accordingly, what are needed, but have not heretofore been developed, are systems and methods for sanitizing stethoscopes and other medical devices that utilize the sanitization cloths, containers, and/or brackets already commonly found in medical facilities, while also reducing the number of manual steps required by medical personnel for use thereof. These and other problems are addressed by the systems and methods of the present disclosure.

### SUMMARY

According to an embodiment of the disclosure, an apparatus for disinfecting stethoscope heads configured to interface with a container having at least one sanitation cloth therein is disclosed. The apparatus comprise a main body comprising a wipe plate and one or more arms provided on opposing sides of the wipe plate, the one or more arms extending away from the main body; a dispensing head extending from the main body, the dispensing head having a dispensing aperture at a first end proximal the main body and a rim at a second end different than the first end, wherein the rim is configured to interface with the container; a motor coupled to at least one roller of the main body to cause a rotary motion of the at least one roller, the at least one roller configured to receive at least one sanitation cloths; an actuator electrically or mechanically coupled to the motor; wherein the at least one sanitation cloth is provided proximal to the wipe plate.

According to another embodiment of the disclosure, a method of sanitizing a stethoscope is disclosed. The method comprises: providing a disinfecting apparatus comprising a main body having a wipe plate and one or more arms provided on opposing sides of the wipe plate, the one or more arms extending away from the main body, a dispensing head extending from the main body, the dispensing head having a dispensing aperture at a first end and a rim at a second end, wherein the rim is configured to interface with a container containing at least one sanitation cloth, a motor coupled to at least one roller of the main body to cause a rotary motion of the at least one roller, the at least one roller configured to receive at least one sanitation cloths, and an actuator electrically or mechanically coupled to the motor; routing the at least one sanitation cloth from the container and over the wipe plate; and engaging the actuator to cause the motor to draw the at least one sanitation cloth over the wipe plate.

The foregoing outlines the features and technical advantages of embodiments of the present disclosure so the detailed description of the disclosure that follows may be better understood. Additional features and advantages of the disclosure will be described hereinafter. It should be appreciated by those skilled in the art the conception and specific embodiments disclosed may be readily utilized as a basis for modifying or designing other structures for carrying out the same purposes of the present disclosure.

### BRIEF DESCRIPTION OF THE DRAWINGS

The foregoing and other aspects of the embodiments disclosed herein are best understood from the following detailed description when read in connection with the accompanying drawings. Included in the drawings are the following figures:

FIG. 1 illustrates a typical sanitization cloths container;

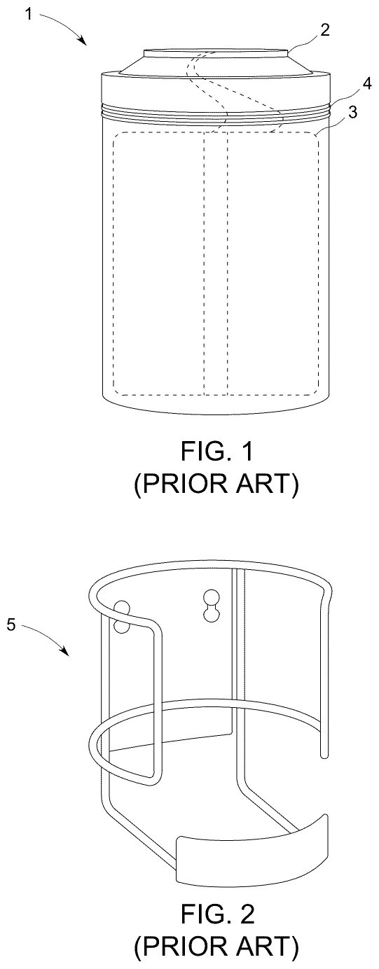

FIG. 2 illustrates a mounting bracket for the sanitization cloths container of FIG. 1;

FIGS. 3A and 3B are front views of a stethoscope head sanitization device, according to embodiments of the present disclosure;

FIGS. 4A and 4B are perspective views of the stethoscope head sanitization device, according to embodiments of the present disclosure;

FIGS. 5A and 5B are top views of the stethoscope head sanitization device, according to embodiments of the present disclosure;

FIGS. 6A-6D illustrate additional perspective views of the stethoscope head sanitization device, according to embodiments of the present disclosure;

FIGS. 7A and 7B illustrate operation of the stethoscope head sanitization device, according to embodiments of the present disclosure;

FIG. 8 is a diagram illustrating another stethoscope head sanitization device, according to the present disclosure, having a sanitization cloths dispensing mechanism; and

FIG. 9 illustrates a dispensing head for the sanitization cloths container, configured for use in connection with the stethoscope head sanitization device, according to embodiments of the present disclosure.

While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the particular form disclosed, but to the contrary, the present disclosure is intended to cover all modifications, equivalents, and alternatives falling within the spirit and scope of embodiments of the present disclosure, as defined by the description.

The headings used herein are for organizational purposes only and are not meant to be used to limit the scope of the description. As used throughout this application, the word “may” is used in a permissive sense (e.g., meaning having the potential to), rather than the mandatory sense (e.g., meaning must). Similarly, the words “include,” “including,” and “includes” mean, “including, but not limited to.” To facilitate understanding of the present disclosure, like reference numerals have been used, where possible, to designate like elements common to the figures.

### DETAILED DESCRIPTION

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B, and C together.

The term “a” or “an” entity refers to one or more of that entity. As such, the terms “a” (or “an”), “one or more” and “at least one” can be used interchangeably herein. It is also to be noted the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation is performed. Human input that consents to the performance of the process or operation is not deemed to be “material.”

In the following description, for the purposes of explanation, numerous specific details are set forth in order to provide a thorough understanding of the embodiments of the disclosure. It is apparent, however, to one skilled in the art the embodiments of the disclosure may be practiced without these specific details or with an equivalent arrangement.

Although various embodiments are described with respect to a stethoscope head sanitization device, it is contemplated the approaches of the various embodiments described herein are applicable to sanitization of any other object as required or recommended.

Reference throughout this specification to “one embodiment” or “an embodiment” means a particular feature, structure, or characteristic described in connection with the embodiment is included in at least one embodiment of the present disclosure. Thus, appearances of the phrases “in one embodiment” or “in an embodiment” in this specification do not necessarily all refer to the same embodiment. Furthermore, the particular features, structures, or characteristics may be combined in any suitable manner in one or more embodiments.

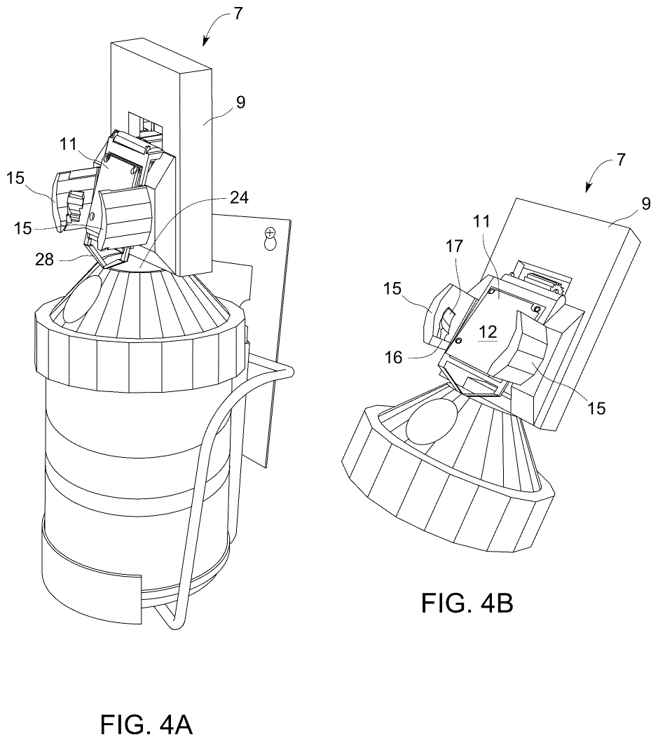

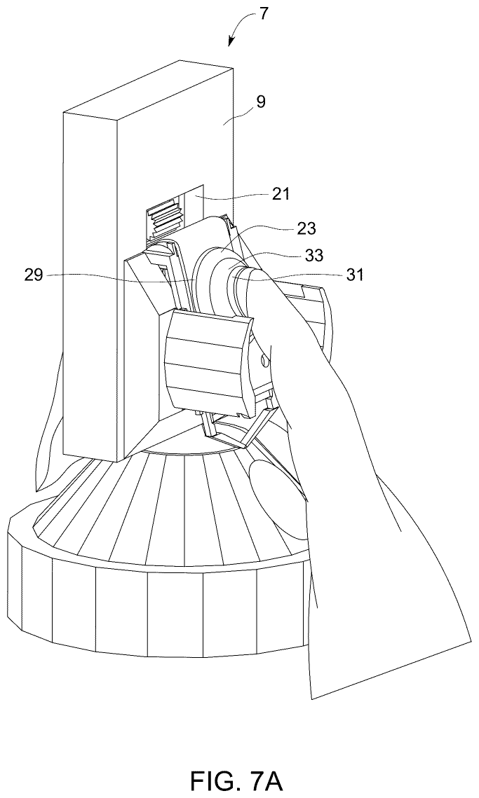

FIGS. 3A and 3B are front views of a stethoscope head sanitization device 7, according to an embodiment. The stethoscope head sanitization device 7 (hereinafter referred to as “sanitization device 7” or “device 7”) has a main body 9 that is mountable or couplable directly on top of the sanitation cloths container 1 as illustrated in FIGS. 3A and 3B. The sanitation device 7 may include a dispensing head 22 with a rim 20 for interfacing with container 1. In some examples, container 1 may be a standard, pre-existing sanitation or disinfecting cloths container 1 commonly used in medical environments. Container 1 may therefore have a standardized opening with coupling members such as threads 4 around a perimeter of the top opening container 1. Other types of coupling members are contemplated and within the scope of the present disclosure. For example, the container 1 may be shaped to form an interference-fit around a perimeter of the opening configured to interface with corresponding interference structures formed on the lid 2 and/or the rim 20. Accordingly, a rim 20 may include complimentary coupling members, such as for example corresponding threads (e.g. threads 26 in FIGS. 6B and 6C). In some examples, rim 20 may be configured with an inner diameter sufficient to ensure a press-fit or friction-fit coupling between the rim 20 and container 1.

With continuing reference to FIGS. 3A and 3B, the main body 9 may have a dispensing head 22 extending from a bottom end of the main body 9. The dispensing head 22 may have a substantially conical shape and configured to ensure sanitation cloths from the container 1 are provided to the main body 9 in a reliable manner while reducing the amount of air exchange with the interior of the container 1 and the sanitation cloths. Dispensing head 22 has an aperture 24 formed at an upper end to allow sanitation cloths 3 to pass therethrough. The dispensing head 22 may be formed integrally with the main body 9 or may be formed separately and attached to the main body 9 using a fastener or coupling means such as threads 26 on rim 20 configured to cooperate with threads 4 on container 1. According to some embodiments of the present disclosure, the main body 9 of the sanitization device 7 is adapted to replace the original lid 2 of the sanitization container 1 and may be configured with the appropriate type of coupling members according to the specific container. Therefore, according to such embodiments, the sanitization device 7 may be secured to the body of the sanitization cloths container 1, eliminating the need to separately mount the main body 9 to a wall.

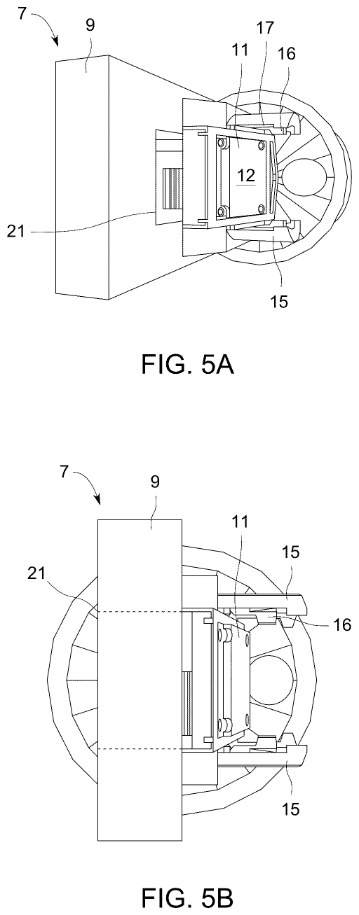

The sanitization device 7 includes a wipe plate 11 that extends from the main body 9. In an embodiment of the present disclosure, the wipe plate 11 is aligned at an angle relative to the main body 9 (e.g., 15, 30, or 45 degrees). Further, the sanitization device 7 may include arms 15 that extend from the main body 9 on either side of the wipe plate 11. The angle of the wipe plate 11 with respect to the main body 9 and the arms 15 is best shown, for example, in FIGS. 4A and 4B. Wipe plate 11 may be sized and configured to receive a sanitation cloth over it to support the sanitation cloth while a stethoscope head is pressed on the sanitation cloth. The wipe plate 11 therefore is configured to be sturdy to accept the pressure of the stethoscope against it while a user is sanitizing the stethoscope head.

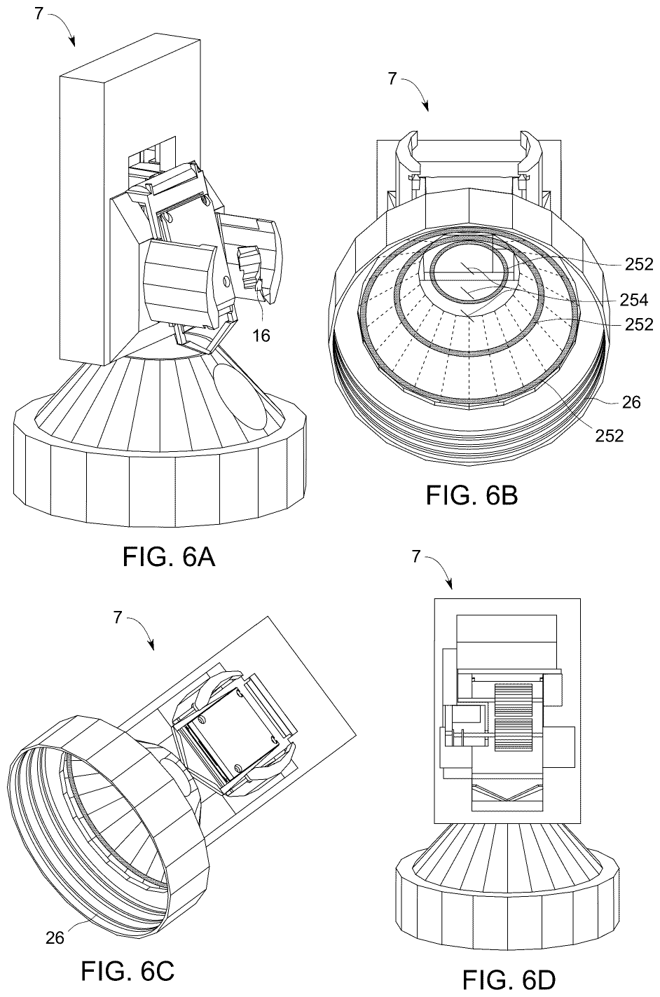

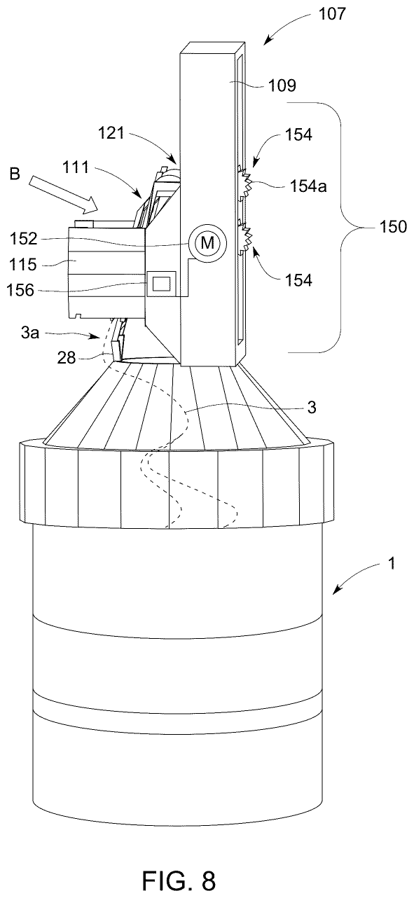

With reference to FIGS. 5A-B, the arms 15 extend from the main body 9 in such a way that a standard stethoscope head can be aligned between the arms 15, thereby ensuring proper alignment with the surface of the stethoscope head. The main body 9, wipe plate 11, and the arms 15 are also illustrated in conjunction with FIGS. 6A-D. In some examples, actuator 12 (which may be the same or similar as actuator 156 in FIG. 8 discussed below) may be provided on, within, or behind the wipe plate 11 to allow for automatic operation as will be described below with respect to FIG. 8. A channel 17 may be formed in the arms 15 such that a stethoscope head can be received therein to ensure alignment with the sanitation cloth and wipe plate 11. In some examples, one or more abutments 16 may be provided on a bottom portion of the arms 15, at a bottom end of channels 17, to receive or contact a portion of stethoscope head. The channels 17 and abutments 16 help to ensure a user does not misalign the stethoscope head and help prevent the stethoscope head from being inserted it too far, such that the entire surface of the stethoscope diaphragm is able to contact the sanitation cloth. A person skilled in the art will appreciate any suitable material such as, but not limited to, plastic, metal, wood, or any composite may be used to construct the sanitization device 7.

With continued reference to FIGS. 5A and 5B, the sanitization device 7 includes a path or opening 21 extending through the main body 9. According to some embodiments of the present disclosure, when preparing the sanitation device 7 for use, the lid 2 is removed from the container 1 and a distal end of the continuous roll of the sanitization cloths is drawn from the container 1 and drawn over a front face of the wipe plate 11. Therefore, according to these embodiments, the front face of the wipe plate 11 is covered with the sanitization cloths 3. According to other embodiments of the present disclosure, the sanitization cloths 3 are first routed through the conveyor structure 28, over the front face of the wipe plate 11 and then drawn through the opening 21 from the front face of the wipe plate 11 to the back of the main body 9. Therefore, according to said other embodiments, the front face of the wipe plate 11 is covered with the sanitization cloths 3. It is noted that in some examples the conveyor structure 28 may be optionally omitted. In other embodiments, the sanitation cloths 3 may alternatively be routed to an upper portion of the main body 9, provided through the rollers 154, through the opening 21, and over the face of the wipe plate 11.

Sanitization cloths 3 are commonly configured as a continuous roll with perforations provided periodically along the length of the continuous roll. After one or more cloths 3 have been arranged as described above, the desired sanitization cloth 3 can be drawn/pulled by applying an upward force to the distal end thereof. Accordingly, the front face of the wipe plate 11 can be continuously covered with the sanitization cloths 3. According to some embodiments of the present disclosure, the distal end of the sanitization cloths 3, after passing over the wipe plate 11, can be routed directly into a refuse bin or the like. The refuse bin (not shown) can be a traditional refuse bin (e.g., positioned on the floor, against the wall, and aligned to receive the distal end of the sanitization cloths 3), the refuse bin can be mounted on the wall adjacent to the device 7 (e.g., positioned to minimize the length of used sanitization cloths 3 extending between the device 7 and the refuse bin), or the refuse bin can be integrally formed with, or attached to, the device 7, such that used sanitization cloths 3 are contained within the device 7 and not exposed to the environment after use. According to such embodiments, a user of the device 7 need not tear used portions of the sanitization cloths 3 from the continuous roll before or after use, thereby minimizing the amount of preparation time required to ready the device 7 for use and thereby encouraging utilization of the device.

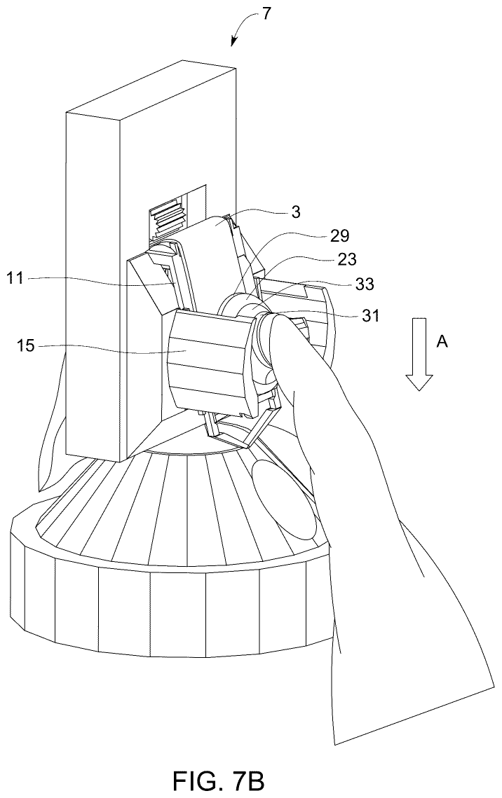

FIGS. 7A and 7B illustrate operation of the sanitization device 7 to sanitize a stethoscope head 23, according to embodiments of the present disclosure. The stethoscope head 23 includes a diaphragm 29, a bell 31 having a smaller diameter than the diaphragm 29, and a drum 33 therebetween. As shown, the wipe plate 11 and the arms 15 of the device 7 are sized and arranged such that a user can press the stethoscope head 23 flat against the exterior surface of the wipe plate 11 and then press the stethoscope head 23 forward along the wipe plate 11 in the direction of arrow A from the position shown in FIG. 7A to the position shown in FIG. 7B. While the diaphragm 29 is in contact with the sanitation cloths 3, a user may move the stethoscope head 23 upward and downward so the diaphragm 29 contacts a larger surface area of the cloths 3, thereby ensuring that sanitation liquid on the cloths 3 contacts and covers the whole surface of the diaphragm 29.

As mentioned above, during preparation the sanitization cloths 3 is positioned over the front face of the wipe plate 11. Accordingly, positioning the diaphragm 29 or drum 33 of the stethoscope head 23 against the wipe plate 11 (with the sanitation cloths 3 provided between the stethoscope head 23, diaphragm 29, and wipe plate 11) and moving the stethoscope head 23 in the direction of arrow A, from the position shown in FIG. 7A to the position shown in FIG. 7B, causes the diaphragm 29 or drum 33 of the stethoscope head 23 to rub against the sanitization cloths 3 thereby wiping and removing of debris and sanitization of the diaphragm 29 and portions of the stethoscope head 23. After the stethoscope head 23 is wiped and sanitized, the used sanitization cloths 3 can be pulled behind the wipe plate 11, so as to draw unused sanitization cloths 3 from the sanitization cloths container 1, and the foregoing steps can be repeated for a second sanitization operation, or for another stethoscope head 23 or medical device.

It should be understood, the used sanitization cloths 3 are also pulled behind the wipe plate 11 prior to a first sanitization operation, so as to draw unused sanitization cloths 3 from the sanitization cloths container 1 and ensure a sufficient amount of sanitization liquid for the sanitization operation is held by the cloths 3 (e.g., ensuring the sanitization liquid has not evaporated prior to sanitization operation). After one or more of the sanitization operations have been completed, the used sanitization cloths 3 can be torn from the continuous roll and discarded, or the used sanitization cloths 3 can be routed into a refuse bin, as discussed above.

FIG. 8 is a diagram illustrating a stethoscope head sanitization device 107 having a dispensing mechanism 150 for drawing the sanitization cloths 3 from the sanitization cloths container 1, according to some embodiments of the present disclosure. It should be understood the device 107 can be substantially similar in structure and functionality to the device 7 discussed above, but for distinctions noted herein.

As shown in FIG. 8, the device 107 includes a main body 109 mountable to the top of a sanitation cloth dispenser, a wipe plate 111 extending from the main body 109 (e.g., at an angle relative to the main body 109), and arms 115 extending substantially perpendicularly from the main body 109 on opposing sides of the wipe plate 111.

The dispensing mechanism 150 of the device 107 may include a motor 152, one or more rollers 154 or the like configured to engage the sanitization cloths 3, and an actuator 156 for controlling operation of the motor 152. It is noted actuator 156 may be the substantially the same as actuator 12 in FIGS. 5A and 5B or may have a different design or actuation action as discussed herein. As illustrated in FIG. 8, the motor 152 is in electrical communication with the actuator 156 and is mechanically coupled to the one or more rollers 154. Rollers 154 may have a plurality of gripping features 154a disposed on the outer circumference thereof, and in some examples the gripping features 154a of the upper roller 154 may be configured to interact with the gripping features 154a of the lower roller 154. In the embodiment of FIG. 8, the sanitization cloths 3 are routed from the container 1, through an opening 121 through upper portion of the main body 109, engaged with (e.g., tensioned about) the one or more rollers 154, routed out through an opening 121, and positioned on the wipe plate 111. Activating the actuator 156 causes the motor 152 to drive the rollers 154, thereby advancing the continuous roll of sanitization cloths 3 and presenting the user with an unused portion of the cloths 3 on the wipe plate 111 for a sanitization operation.

According to embodiments of the present disclosure, the actuator 156 can be a button, switch, proximity sensor, pressure sensor, light sensor, or any other means for detecting the stethoscope head 23 positioned on the wipe plate 111. Actuator 156, which may be the same as the actuator 12 in FIG. 5A, may be provided on top of the wipe plate 111, integrally formed with the wipe plate 111, provided behind the wipe plate 111, and the like. For example, the actuator 156 may comprise a button engaged by pressure applied to the wipe plate 111 in the direction of arrow B.

In other examples, the actuator 156 may be a photodetector configured to detect a drop in light to indicate a stethoscope head 23 has been placed over the sanitization cloths 3 and actuator 156. An aperture (not shown) may be provided in the wipe plate 111 to allow for a photodetector to determine when a stethoscope head 23 has been positioned over the sanitization cloths 3. The actuator 156 can be configured to cause the device 107 to cause the motor 152 to advance a predetermined length of the sanitization cloths 3 prior to a sanitization operation (that is, in response to actuation of the actuator 156, the motor 152 may be configured to automatically rotate a certain number of rotations to ensure unused sanitation cloths 3 is provided over the wipe plate 111. Additionally, or alternatively, actuator 156 can be configured to cause the device 107 to continuously advance the sanitization cloths 3 as long as pressure applied to the wipe plate 111 in the direction of arrow B, thereby allowing the user to determine the length of sanitization cloths 3 dispensed from the container 1 and also allowing the user to determine the length and intensity of the sanitization operation.

In some embodiments of the present disclosure, the wipe plate 111 is adapted to pivotably engage the actuator 156, such as for example the wipe plate 11 may pivot about a hinged attachment point at the upper part of the wipe plate 11 proximal to the opening 121. Any type of hinged mounting design may be used to allow pivotable motion of the wipe plate 11 by a predetermined amount to actuate the actuator 156. For example, as shown in FIG. 8, the actuator 156 may be positioned adjacent to a rear side of the wipe plate 111 and may comprise a pressure sensor (e.g., strain gauge or the like). According to such embodiments, when the stethoscope head 23 is applied to wipe plate 111, a pivot motion of the wipe plate 111 causes the actuator 156 to be activate, thereby controlling the motor 152 and rollers 154 of the dispensing mechanism 150 to advance the sanitization cloths 3, drawing the roll of sanitization cloths 3 between the stethoscope head 23 and the wipe plate 111, and sanitizing the stethoscope head 23.

According to some embodiments of the present disclosure, the stethoscope head 23 can be stationary, applied onto the wipe plate 111, while the sanitization cloths 3 are drawn along the wipe plate 111 by the dispensing mechanism 150. Alternatively, the user may move the stethoscope head 23 while the dispensing mechanism 150 draws the sanitization cloths 3 from the container 1 and along the wipe plate 111. For example, the user may move the stethoscope head 23 in an opposite direction to the dispensing to speed up the sanitization process.

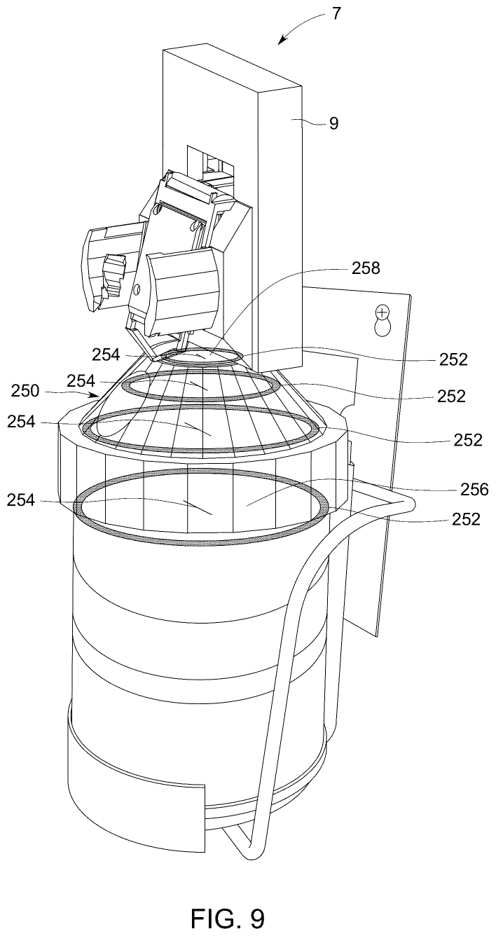

FIG. 9 illustrates a dispensing head 250 formed integrally with the main body 9 and/or adapted for attachment to one or more of the sanitization devices of the present disclosure (e.g., device 7 and/or device 107). The dispensing head 250 can be configured to minimize the amount of sterilization fluid lost through evaporation and to assist with routing the sanitization cloths 3 from the container 1 to the sanitization devices of the present disclosure. It will be understood by those of ordinary skill in the art that existing designs of the lid 2 of a container 1 may include an aperture for drawing the sterilization cloths 3 from an interior of the container 1 and the lid 2 is configured to seal the aperture after the user has dispensed a desired amount of sanitization cloths 3, such that sterilization fluid within the container 1 is prevented from escaping the container 1 through the aperture due to evaporation. However, according to some embodiments of the present disclosure, it may not be possible to close the lid 2 and seal the aperture, because doing so may prevent or inhibit the sanitization devices of the present disclosure from drawing the continuous roll of sanitization cloths 3 from the container 1. Accordingly, the dispensing head 250 has a rim at a lower end which is configured to secure the dispensing head 250 to the sanitization cloths container 1, as discussed above, to enable dispensing of sanitization cloths 3, while preventing evaporation of the sanitization fluid from the container 1. According to embodiments of the present disclosure the dispensing head 250 can be configured to be releasably secured to the body of the container 1 (e.g., replacing the lid 2) using threaded engagements, press-fits, interference-fits, and the like, as discussed above.

As illustrated in FIG. 9, the dispensing head 250 can include diaphragms 252 disposed at one or more of a bottom opening 256 of the dispensing head 250, a top opening 258 of the dispensing head 250, and/or throughout the dispensing head 250. Diaphragms 252 may be formed of a flexible material (e.g., rubber, a pliable plastic, and the like) and provided with openings 254 to allow the sanitization cloths 3 to pass therethrough while minimizing the amount of sanitization fluid that can escape from the container 1. Diaphragms 254 and openings 254 in some examples may be shaped to prepare, align, and/or flatten the sanitation cloths 3 for proper routing through conveyor structure 28, rollers 154, and wipe plate 11.

With continued reference to FIG. 9, the dispensing head 250 may have a conical shape and cause the sanitization cloths 3 to be drawn around the cone in a gradually reducing circumference with the top opening 258 being directed to a routing path of the sanitization device 7 or sanitation device 107. For example, as discussed above, a leading edge 3a (see FIG. 8) of the sanitization cloths 3 may be drawn from the dispensing head 250, through conveyor structure 28, over the wipe plate 11, and through the opening 121 in the main body 9. Alternatively, the leading edge 3a of the sanitization cloths 3 may be drawn from the dispensing head 250 first through the opening 121 in the main body 9 and over the wipe plate 11 of the device 7. A person skilled in the art will appreciate different angles of the conical shape may be used based on the length or size of the sanitization cloths 3 to be drawn and/or the location of the dispensing head 250 and container 1 relative to the sanitization devices of the present disclosure.

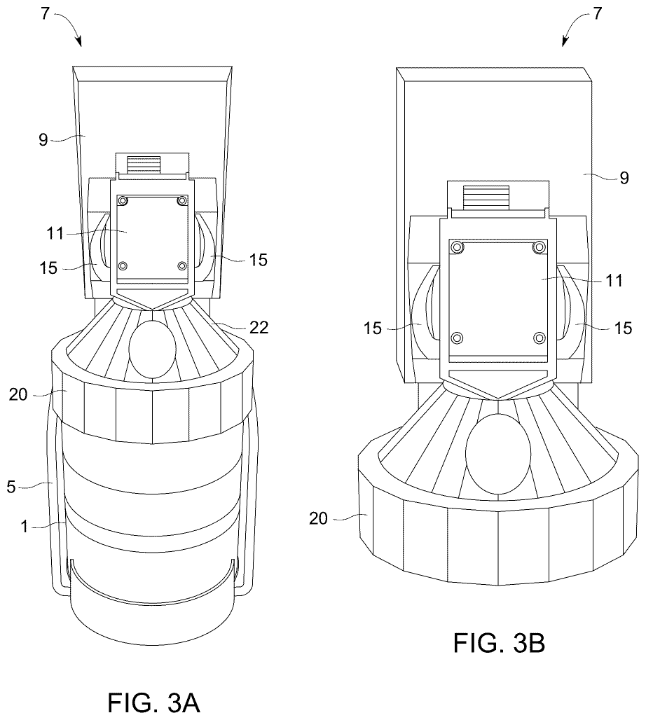

When preparing the sanitation device 7 for use, the sanitation device 7 is coupled to a container 1 as described in the present disclosure and illustrated in FIGS. 2-9. A user may draw a sanitation cloth 3 through the dispensing head 250, including through the one or more diaphragms 252 and openings 254, if provided. The sanitation cloth 3 may be drawn through the top opening 258 of the dispensing head 250 and through the conveyor device 28, if a conveyor device 28 is provided. A user may provide the sanitation cloth 3 over the wipe plate 11, through the opening 121, and feed the sanitation cloth 3 between the rollers 154 (and the roller gripping members 154a). Once provided in between the rollers 154, the motor 152 may operably draw additional sanitation cloths 3 through the device 7 in response to actuation by the user of the actuator 156.

Accordingly, once the sanitation device 7 is coupled to the container 1 and the sanitation cloths 3 are engaged with the rollers 154, the sanitation device 7 is ready for use. During use and as illustrated in for example FIGS. 7A, 7B and 8, the user presses the surface of a stethoscope head 23 against the sanitation cloth 3 and wipe plate 11 in the direction of the arrow B in FIG. 8. A user may then slide the stethoscope head 23 in the direction of the arrow A in FIG. 7B, which may be a direction substantially parallel to the wipe surface of the wipe plate 11. The user slides the stethoscope head 23 in the direction of arrow A until the stethoscope head 23 is provided in the channels 17 and rests on the abutments 16, if abutments 16 and/or channels 16 are provided. The sanitation cloth 3 is thus provided between the stethoscope head 23 and the surface of the wipe plate 11. In some examples, the actuator 156 may be activated when the stethoscope head 23 is pressed against the sanitation cloths 3 (and thus against the wipe plate 11). For example, if an actuator 156 is a photodetector, capacitive sensor, proximity detector, and the like, then the presence of the stethoscope head 23 is detected when the user provides the stethoscope head 23 against the sanitation cloth 3 and wipe plate 11. Therefore, when such an actuator 156 detects the stethoscope head 23 the actuator 156 may actuate the motor 152 causing rollers 154 to draw additional sanitation cloths 3 through the device 7 while the stethoscope head 23 is pressed against the sanitation cloth 3 provided over the surface of the wipe plate 11. In some examples, the actuator may be positioned such that it actuates the motor 152 when the stethoscope head is provided in the channels 17 and against the abutments 16 to ensure proper positioning of the stethoscope head 23 against the wipe surface with the sanitation cloth 3 therebetween. In other examples, the actuator 156 may activate the motor 152 immediately upon detecting the presence of the stethoscope head 23 against the sanitation cloth 3 and/or wipe plate 11.

In another example, as discussed herein, if the wipe plate 11 is pivotably mounted on the main body 9 such as mounted at a hinged attachment point at the top of the wipe plate 11 proximal to the opening 121, the user may actuate the actuator 156 by pressing the stethoscope head 23 against the wipe plate 11 surface in a direction of the arrow B in FIG. 8 (which may be substantially orthogonal to the surface of the wipe plate 11). By applying a predetermined pressure in direction of the arrow B against the surface of the wipe plate 11, the actuator 156 may actuate the motor 152 causing rollers 154 to draw additional sanitation cloths 3 through the device 7 while the stethoscope head 23 is pressed against the sanitation cloth 3 provided over the surface of the wipe plate 11.

As discussed above, the motor 152 may be configured to draw a predetermined amount of sanitation cloths 3 through the rollers 154 based upon actuation of the actuator 156. The action of the rollers 154 draws fresh sanitation cloths 3 through the device 7 thereby sanitizing the stethoscope head 23. In other configurations, upon actuation of the actuator 156 by the user the motor may continuously draw sanitation cloths 3 until the user removes the stethoscope head and the actuator 156 is no longer activated.

The sanitization devices of the present disclosure may provide several advantages over the conventional techniques. The sanitization devices of the present disclosure may require less manual control or handling of the sanitization cloths 3 resulting in better sanitization/disinfection. Moreover, the dispensing of the sanitization cloths 3 may be automated to further improve effectiveness of a sanitization operation. Furthermore, the sanitization devices of the present disclosure may be conveniently mounted directly on top of pre-existing sanitization cloths containers 1, so a separate mounting arrangement may not be required.

While the disclosure has been described in connection with a number of embodiments and implementations, the disclosure is not so limited but covers various obvious modifications and equivalent arrangements, which fall within the purview of the appended claims. Although features of the disclosure are expressed in certain combinations among the claims, it is contemplated these features can be arranged in any combination and order.

The exemplary embodiments of this present disclosure have been described in relation to sanitizing stethoscope heads. However, to avoid unnecessarily obscuring the present disclosure, the preceding description omits a number of known structures and devices. This omission is not to be construed as a limitation of the scope of the present disclosure. Specific details are set forth by use of the embodiments to provide an understanding of the present disclosure. It should however be appreciated that the present disclosure may be practiced in a variety of ways beyond the specific embodiments set forth herein.

A number of variations and modifications of the present disclosure can be used. It would be possible to provide for some features of the present disclosure without providing others.

The present disclosure, in various embodiments, configurations, and aspects, includes components, methods, processes, systems and/or apparatus substantially as depicted and described herein, including various embodiments, sub-combinations, and subsets thereof. Those of skill in the art are able to understand how to make and use the present disclosure after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and/or reducing cost of implementation.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more embodiments, configurations, or aspects for the purpose of streamlining the disclosure. The features of the embodiments, configurations, or aspects may be combined in alternate embodiments, configurations, or aspects other than those discussed above. This method of disclosure is not to be interpreted as reflecting an intention the present disclosure requires more features than are expressly recited in each claim. Rather, as the following description reflect, inventive aspects lie in less than all features of a single foregoing disclosed embodiment, configuration, or aspect. Thus, the following description are hereby incorporated into this Detailed Description, with each claim standing on its own as a separate embodiment of the present disclosure.

Moreover, though the description of the present disclosure has included description of one or more embodiments, configurations, or aspects and certain variations and modifications, other variations, combinations, and modifications are within the scope of the present disclosure, e.g., as may be within the skill and knowledge of those in the art, after understanding the present disclosure. It is intended to obtain rights that include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An apparatus for disinfecting stethoscope heads configured to interface with a container having at least one sanitation cloth therein, the apparatus comprising:
a main body comprising a wipe plate and one or more arms provided on opposing sides of the wipe plate, the one or more arms extending away from the main body;
a dispensing head extending from the main body, the dispensing head having a dispensing aperture at a first end proximal the main body and a rim at a second end different than the first end, wherein the rim is configured to interface with the container;
a motor coupled to at least one roller of the main body to cause a rotary motion of the at least one roller, the at least one roller configured to receive at least one sanitation cloths;
an actuator electrically or mechanically coupled to the motor; and
wherein the at least one sanitation cloth is provided proximal to the wipe plate.

2. The apparatus of claim 1, wherein the dispensing head is substantially conical with a larger diameter at the second end proximal the rim and a smaller diameter at the first end.

3. The apparatus of claim 2, wherein the dispensing head further comprises one or more diaphragms disposed on an inner surface between the dispensing aperture and the rim, the one or more diaphragms each comprise an aperture configured to allow the at least one sanitation cloth to pass therethrough.

4. The apparatus of claim 1, wherein the wipe plate further comprises a sanitizing surface for receiving the at least a portion of the at least one sanitation cloth.

5. The apparatus of claim 1, wherein the wipe plate is provided on a hinged support such that the wipe plate can rotate between a first position and a second position, wherein in the second position the actuator activates the motor.

6. The apparatus of claim 4, wherein the at least one sanitation cloth is provided between the wipe plate and a diaphragm of the stethoscope such that the at least one sanitation cloth is in direct contact with the diaphragm.

7. The apparatus of claim 1, wherein the rim further comprises a coupling member for coupling the dispensing head to the container, wherein the rim couples the dispensing member to the container using a threaded coupling, a press-fit coupling, a friction fit coupling, and/or an interference fit coupling.

8. The apparatus of claim 1, wherein the at least one roller provides tension to the at least one sanitation cloth.

9. The apparatus of claim 8, wherein the at least one roller is configured to grip the at least one sanitation cloth and, based on the rotary motion of the motor, draw the at least one sanitation cloth across the wipe plate.

10. The apparatus of claim 1, wherein when the actuator is actuated, the motor is configured to run for a predetermined amount of time to draw a predetermined amount of the at least one sanitation cloth through the apparatus.

11. The apparatus of claim 1, wherein when the actuator is actuated, the motor is configured to run continuously.

12. The apparatus of claim 1, wherein the actuator is selected from the group consisting of a photodetector, a button, a pressure-sensitive actuator, or a proximity sensor.

13. The apparatus of claim 1, wherein the wipe plate is movable, and the actuator is activated by the movement of the wipe plate such that pressing the stethoscope head onto the wipe plate causes the activation of the actuator.

14. A method of sanitizing a stethoscope, the method comprising:
providing a disinfecting apparatus, the disinfecting apparatus comprising:
a main body comprising a wipe plate and one or more arms provided on opposing sides of the wipe plate, the one or more arms extending away from the main body;
a dispensing head extending from the main body, the dispensing head having a dispensing aperture at a first end and a rim at a second end, wherein the rim is configured to interface with a container containing at least one sanitation cloth;
a motor coupled to at least one roller of the main body to cause a rotary motion of the at least one roller, the at least one roller configured to receive at least one sanitation cloths; and
an actuator electrically or mechanically coupled to the motor;
routing the at least one sanitation cloth from the container and over the wipe plate; and
engaging the actuator to cause the motor to draw the at least one sanitation cloth over the wipe plate.

15. The method of claim 14, wherein engaging the actuator comprises pressing a stethoscope against wipe plate or pressing the stethoscope against at least one sanitation cloth, wherein the at least one sanitation cloth is positioned over the wipe plate.

16. The method of claim 14, wherein the actuator is selected from the group consisting of a photodetector, a button, a pressure-sensitive actuator, or a proximity sensor.

17. The method of claim 16, wherein in response to engaging the actuator, the motor operates for a predetermined amount of time to draw a predetermined amount of the at least one sanitation cloth through the apparatus.

18. The method of claim 16, wherein in response to engaging the actuator, the motor operates continuously while the actuator is engaged to draw a continuous amount of the at least one sanitation cloth through the apparatus.

19. The method of claim 14, wherein the dispensing head further comprises at least one diaphragm provided on an inner surface between the dispensing aperture and the rim.

20. The method of claim 19, wherein the one or more diaphragms each comprise an aperture configured to allow the at least one sanitation cloth to pass therethrough.
